(19) United States
(12) Patent Application Publication
(10) Pub. No.: US 2019/0263891 A1
(43) Pub. Date: Aug. 29, 2019

(54) PROCESS FOR ISOLATING BIOACTIVE BIOMOLECULES FROM ANIMAL BY-PRODUCTS

(71) Applicants: Beth Mason, Sydney (CA); Zied Khiari, Lethbridge (CA)

(72) Inventors: Beth Mason, Sydney (CA); Zied Khiari, Lethbridge (CA)

(21) Appl. No.: 16/286,969

(22) Filed: Feb. 27, 2019

Related U.S. Application Data
(60) Provisional application No. 62/636,414, filed on Feb. 28, 2018.

(51) Int. Cl. C07K 14/78; C07K 1/14; C08H 1/06; C08B 37/00; C08B 37/08

(52) U.S. Cl. CPC C07K 14/78; C07K 1/14; C08B 37/0066; C08B 37/0069; C08B 37/0072; C08H 1/06

(57) ABSTRACT

A process for producing a plurality of biomolecule products from by-products of animal food processing is described. The process includes the steps of mixing the by-products with one or more digestive enzymes in the presence of an acid to promote hydrolysis of the by-product to release the biomolecules, thereby providing a hydrolysis mixture. The hydrolysis mixture is subjected to a density-based fractional separation, thereby providing an oil fraction, a liquid fraction and a solid fraction. Then the liquid fraction is separated from the oil and solid fractions and filtered with a molecular mass cutoff filter, thereby providing a peptide product and a glycosaminoglycan product. The oil fraction may be further refined to provide an oil product and the solid fraction may be further processed to provide bone-derived products such as gelatin, ossein and collagen.

### CROSS-REFERENCE TO RELATED APPLICATIONS

This application claims priority from U.S. Provisional Application Ser. No. 62/636,414, filed on Feb. 28, 2018, the entire disclosure of which is incorporated herein by reference.

### FIELD OF THE INVENTION

The invention relates to the field of functional food ingredients and provides processes for isolation of bio-active biomolecules from animal by-products which are generally been considered as waste products.

### BACKGROUND

The fish and meat processing industries generate significant amounts of by-products which are generally regarded as wastes and discarded (Khiari et al., 2013, Food Chemistry, 139:347-354, incorporated herein by reference in entirety). If this material is not properly treated, it can lead to environmental and health issues. In many cases, this waste is either dumped into landfill or composted.

Fish and meat wastes such as viscera, bones, heads, skin, cartilage, connective tissues and appendages) constitute an important source of bioactive compounds such as collagen and glycosaminoglycans (Khiari et al., 2014, Poultry Science, 93: 2347-2362, incorporated herein by reference in entirety).

The functional food ingredient sector is rapidly growing and expected to continue to attract further investment. For example, the market value of gelatin was US$1.77 billion in 2011 and was projected to reach $2.79 billion (USD) in 2018. The major producers globally produce 450.7 kilotons of gelatin.

Fish oil, which has long been recognized as a health-promoting ingredient, remains an important commodity which had a market value of $1.1 billion (USD) in 2011 and was projected to reach $1.7 billion (USD) in 2018. The major producers globally produce of 1,130 kilotons of fish oil.

Collagen is the most abundant protein of animal origin, representing approximately 30% of total animal protein. Collagen is found in all connective tissue, including bones and skin. It has been reported that the oral ingestion of hydrolyzed collagen (also termed collagen peptides) promotes collagen synthesis in the skin and increases the size of collagen fibrils in the dermis (Matsumoto et al., 2006, ITE Letters on Batteries, New Technologies and Medicine, 7:386-390, incorporated herein by reference in entirety). Results from clinical studies have indicated that the daily intake of collagen peptides improves the hydration of skin and prevents wrinkle formation (Borumand & Sibilla, 2015, Journal of Medical Nutrition and Nutraceuticals, 4:47-53, incorporated herein by reference in entirety).

The industrial production of collagen peptides requires two separate operations. In the first step, gelatin is extracted and purified, and in the second step collagen peptides are enzymatically produced, sterilized, and finally dried. The extraction of gelatin is time and energy consuming. For instance, the production of type A gelatin (i.e., acid pre-treated by-products) requires up to 30 h of pretreatment, whereas type B gelatin (i.e., alkali pre-treated by-products) needs a longer pretreatment period. For both gelatin types, the extraction is performed in 4 to 5 successive batch operations, each lasting from 4 to 8 h using elevated temperatures from 55 to 100° C. (Schrieber & Gareis, 2007, Gelatine handbook: Theory and industrial practice. Wiley-VCH GmbH & Co., Weinheim, Germany, incorporated herein by reference in entirety).

Collagen peptides and glycosaminoglycans are two emerging functional ingredients in supplements for healthy skin and improved cartilage and joint function. According to the latest estimations, the collagen peptide market was valued at $0.7 billion (USD) in 2013 and is expected to reach $1.1 billion (USD) by 2020.

Glycosaminoglycans are acidic polysaccharides found with high concentration in cartilaginous tissues (Nakano et al., 2012, Process Biochemistry, 47:1909-1918 incorporated herein by reference in entirety). Glycosaminoglycans have a wide range of applications in the pharmaceutical, cosmetic and food industries. In health food markets, glycosaminoglycans are popular dietary supplement for joint care. Oral administration of glycosaminoglycans has been reported to be beneficial in the treatment of osteoarthritis. Bovine nasal and tracheal cartilage and shark cartilage are the most common sources of commercial glycosaminoglycans. However, bovine tissues are suggested to have potential risks of infectious diseases (e.g. bovine spongiform encephalopathy), and the supply of shark cartilage is limited.

The market for glycosaminoglycan (mainly chondroitin sulfate) is undergoing rapid development and is estimated to reach $0.8 billion (USD) by 2021. Global production of chondroitin sulfate is currently at about 10.39 kilotons and is projected to attain 12.98 kilotons in 2021.

The market for bioactive molecules as supplements to promote joint health, among which collagen peptides and glycosaminoglycans are prominent examples, is the most rapidly growing sector among the nutraceutical ingredient market. There is a substantial demand for these supplements mainly in Japan, the United States and Europe. These functional biomolecules are also formulated in pet food and nutraceutical pet formulations. Both collagen peptides and glycosaminoglycans are among the most established pet supplements. In the US alone, retail sales of pet supplements and nutraceutical pet treats reached $1.3 billion (USD) in 2012 and has an annual growth rate of 1.4%. Sales of pet supplements and other natural and organic pet supplies are projected to grow by 3 to 5% on an annual basis which will result in a market value of US$1.6 billion in 2017.

Soluble peptides and glycosaminoglycans are used in liquid feeding systems for animal farming. The fat (fish oil/poultry fat) are incorporated in poultry feeds or juvenile fish farming. Ossein is used as a starting material for the production of gelatin, which is a highly demanded multifunctional bio-polymers. With minimal additional processing operations (purification and concentration) the obtained bio-products can be used as functional ingredients for novel pet food products (e.g. fortified pet food). Food, cosmetic, nutraceutical and pharmaceutical applications require higher quality products. To achieve this requirement, further processing operations are needed which substantially add cost to the entire process.

Glycosaminoglycan production involves the isolation of raw cartilage followed by a hydrolysis step that breaks down the proteoglycan core. Subsequently, the proteins are eliminated and the glycosaminoglycans are recovered and purified. The hydrolysis is commonly performed using alkaline treatment with high concentrations of NaOH, urea or guanidine HCl. The deproteinization is achieved by trichloroacetic acid precipitation and the purification is carried out by means of gel filtration and/or ion-exchange and size-exclusion chromatography (Vazquez et al., 2013, Marine Drugs, 11: 747-774, incorporated herein by reference in entirety).

For fish oil, the primary extraction method is based on a wet pressing technique. The fish material is first heated to elevated temperatures (about 95° C.) which breaks down the tissue material and separates water and oil from proteins. The oil is then separated and recovered by centrifugation.

Various enzymatic procedures have been developed for extracting chondroitin sulfate, the most abundant glycosaminoglycan. For example, JP2001247602A, incorporated herein by reference in its entirety, describes the use of pronase at pH 7.8 and 37° C. for 3 h to extract chondroitin-sulfate from salmon. U.S. Pat. No. 9,347,081, incorporated herein by reference in its entirety, describes the use of pepsin (0.6-1.0%) at pH 3.0-3.5 and 45-55° C. for 20-28 h to extract chondroitin-sulfate from cartilage. CN102850466A, incorporated herein by reference in its entirety, describes the use of pepsin (proportion 1000:0.5) for extraction of chondroitin-sulfate from yak bone at 45° C. for 1.5 h. CN102690372A, incorporated herein by reference in its entirety, describes extraction of chondroitin sulfate from chicken bones using heat and pressure treatments at 125-128° C. and 0.28-0.3 MPa for 1.2-1.5 h.

Other processes describe physical methods for the preparation of glycosaminoglycans. For example, EP1614697A1, incorporated herein by reference in its entirety, describes a process based on pulverization and aqueous solubilization.

There continues to be a need for more efficient isolation of various bioactive biomolecules.

### SUMMARY

One aspect of the invention is a process for producing a plurality of biomolecule products from by-products of animal food processing. The process comprises the steps of mixing the by-products with one or more digestive enzymes in the presence of an acid to promote hydrolysis of the by-product to release the biomolecules, thereby providing a hydrolysis mixture; subjecting the hydrolysis mixture to a density-based fractional separation, thereby providing an oil fraction, a liquid fraction and a solid fraction; separating the liquid fraction from the oil and solid fractions and filtering the liquid fraction with a molecular mass cutoff filter, thereby providing a peptide product and a glycosaminoglycan product.

Certain embodiments of the process may further include a step of processing bone tissue contained in the solid fraction to generate one or more of or a combination of: a collagen product, a gelatin product and an ossein product. The process may include homogenization of the by-products prior to or during the mixing step.

Some embodiments of the process may further include a step of processing the oil to provide a refined oil product.

The digestive enzymes may be provided in the form of substantially intact biological tissues. In this context, “substantially intact” means that the biological tissue of the by-products is generally in the same form when discarded from the food processing process. For example, after harvesting of fish filets as a primary food product, the remaining parts of the fish would be considered as by-products and would simply be fed into the process for producing the plurality of biomolecule products without any substantial intermediate processing steps. However, it is to be understood that some incidental modification or damage to the biological tissue of the by-product could occur during harvesting of the primary food product and the biological tissue would still be considered to be substantially intact. In some embodiments, the biological tissues are of substantially intact organs, meaning that once the organs are removed, they would simply be fed into the process for producing the plurality of biomolecule products without any intermediate processing steps, likewise substantially intact organs, such as digestive tract organs, pancreas, liver and salivary glands may have sustained some damage during harvesting of the primary food product, yet would still be considered substantially intact. Such biological tissues include digestive enzymes including endogenous proteases and peptidases which digest proteins, amylases, which digest polysaccharides, lipases, which digest lipids and nucleases which digest nucleic acids.

In some embodiments, the biological tissues comprise undifferentiated viscera, meaning that the organs of the viscera are not specifically selected or sorted, but instead are collected as a mass for feeding into the process. The undifferentiated viscera may include digestive tract organs, pancreas, liver and salivary glands, for example.

In some embodiments, the digestive enzymes used in the process are provided in the form of crude formulations or purified/commercially available formulations which are prepared from microorganisms such as bacteria and fungi, or prepared from plant or animal sources.

The animal by-products may comprise any one of or any combination of: bones, skin, organs, cartilage, connective tissues and appendages from fish, poultry or mammals.

In certain embodiments, the acid is an organic acid or a mineral acid which is directly added to obtain the hydrolysis mixture. The organic acid may be a low molecular weight organic acid such as formic acid, ethanoic acid, propanoic acid, butanoic acid and lactic acid, for example.

In other embodiments, the acid is produced during fermentation by one or more species or strains of acid-producing bacteria, such as lactic acid producing bacteria. In these embodiments, the fermentation process includes addition of a carbohydrate to promote acid production by the bacteria.

In some embodiments, the density-based fractional separation is centrifugation and the ultrafiltration step uses a molecular mass cutoff filter with a molecular mass cutoff between about 5 kDa to about 15 kDa.

In some embodiments, the glycosaminoglycan product comprises any one of or a combination of: hyaluronic acid, chondroitin sulfate, dermatan sulfate and keratan sulfate.

Another embodiment of the invention is a process for producing a plurality of biomolecule products from by-products of animal food processing which includes the steps of mixing the by-products with undifferentiated viscera in the presence of an acid to promote hydrolysis of the by-product to release the biomolecules, thereby providing a hydrolysis mixture; subjecting the hydrolysis mixture to a density-based fractional separation, thereby providing an oil fraction, a liquid fraction and a solid fraction; and separating the liquid fraction from the oil and solid fractions and filtering the liquid with a molecular mass cutoff filter, thereby providing a peptide product and a glycosaminoglycan product. In some embodiments the undifferentiated viscera are obtained from fish as fish viscera are easily obtained at fish processing facilities and expected to provide a reliable source of digestive enzymes for the process.

### BRIEF DESCRIPTION OF THE DRAWINGS

Various objects, features and advantages of the invention will be apparent from the following description of particular embodiments of the invention, as illustrated in the accompanying drawings.

FIG. 1 is a process flow diagram illustrating one process embodiment 100 of the invention which includes acid-assisted hydrolysis.

FIG. 2 is a process flow diagram illustrating another process embodiment 200 of the invention which includes microbial-assisted hydrolysis.

FIG. 3A is a sodium dodecyl sulfate polyacrylamide gel electrophoresis protein profile obtained from an acid-assisted process incorporating steps shown in FIG. 1, showing the degradation of high molecular weight proteins in fish, poultry and meat by-products into smaller peptides with hydrolysis promoted by formic acid. The centrifugation separates bones and fat from the soluble fraction. The ultrafiltration separates peptides (less than 10 kDa) from glycosaminoglycans (greater than 10 kDa). The glycosaminoglycans are recovered after ethanol precipitation. The hydrolysis was followed at different time points up to 72 hours. Lane 1 is a set of molecular weight markers (14.4-116 kDa); lane 2 is a sample prior to hydrolysis; lane 3 is the sample after 6 hours of hydrolysis; lane 4 is the sample after 12 hours hydrolysis; lane 5 is the sample after 24 hours of hydrolysis; lane 6 is the sample after 48 hours of hydrolysis and lane 7 is the sample after 72 hours of hydrolysis.

FIG. 3B is a sodium dodecyl sulfate polyacrylamide gel electrophoresis protein profile obtained from the microbial-assisted process incorporating steps shown in FIG. 2, showing the degradation of high molecular weight proteins in fish, poultry and meat by-products into smaller peptides with hydrolysis promoted by lactic acid. The centrifugation separates bones and fat from the soluble fraction. The ultrafiltration separates peptides (less than 10 kDa) from glycosaminoglycans (greater than 10 kDa). The glycosaminoglycans are recovered after ethanol precipitation. The hydrolysis was followed at different time points up to 72 h Lane 1 is a set of molecular weight markers (14.4-116 kDa); lane 2 represents a sample prior to hydrolysis; lane 3 is the sample after 6 hours of hydrolysis; lane 4 is the sample after 12 hours hydrolysis; lane 5 is the sample after 24 hours of hydrolysis; lane 6 is the sample after 48 hours of hydrolysis and lane 7 is the sample after 72 hours of hydrolysis.

FIG. 4A is a cellulose acetate electrophoresis profile of glycosaminoglycan patterns obtained from the acid-assisted process incorporating steps of FIG. 1 with fish, poultry and meat by-products, showing the type of glycosaminoglycan (sulfated and/or non-sulfated) present in the precipitated final sample. Lane 1 represents a mixture of glycosaminoglycan standards comprising hyaluronic acid, dermatan sulfate and chondroitin sulfate; lane 2 is a sample of extracted glycosaminoglycans. The vertical arrow indicates the direction of the current (from negative to positive).

FIG. 4B is a cellulose acetate electrophoresis profile of glycosaminoglycan patterns obtained from the microbial-assisted process incorporating steps of FIG. 2 with fish, poultry and meat by-products, showing the type of glycosaminoglycan (sulfated and/or non-sulfated) present in the precipitated final sample. Lane 1 represents a mixture of glycosaminoglycan standards comprising hyaluronic acid, dermatan sulfate and chondroitin sulfate; lane 2 is a sample of extracted glycosaminoglycans. The vertical arrow indicates the direction of the current (from negative to positive).

### DETAILED DESCRIPTION

### Rationale and Overview

There is a need for simplified processes for isolation of commercially important bioactive molecules such as glycosaminoglycans and collagen. Isolation processes tend to be focused on individual biomolecules. Current industrial processes for the preparation of glycosaminoglycans and hydrolyzed collagen are unsustainable and non-ecological. They are heavily based on the use of harsh chemical treatments and costly enzymatic reactions which consume significant amounts of water and energy and generate large amounts of effluents requiring significant treatment before discharge. Extraction of glycosaminoglycans and collagen peptides also requires several pre-treatment operations, including separation of cartilage and collagen, fat removal, and protein denaturation to facilitate the extraction and solubilization of these bio-molecules.

Embodiments of the invention described herein provide economic and ecological benefits by using endogenous digestive enzymes of tissues of by-products which are typically considered as waste material. The processes described herein are capable of producing a plurality of different classes of bio-active biomolecule products from a complex matrix of fish, poultry and meat waste biomass without the need for a defatting step or alkaline and thermal pre-treatments as required by the current processing techniques. These process embodiments use animal by-products such as fish, poultry and mammal wastes (skins, cartilage, connective tissue, appendages and bones) as a feed stock to generate the stream of bio-active biomolecule products. The inventors recognized that among the by-products of animal processing are tissues which are typically found in organs such as digestive tract organs, pancreas, salivary glands, and liver, among others, which include various classes of digestive enzymes such as proteases, peptidases, amylases, lipases and nucleases which could be used to promote the extensive hydrolysis required to liberate the desired bio-active biomolecules from their matrices. Development of a process using raw tissues containing digestive enzymes is be expected to be beneficial in respect to use of otherwise low-value by-products as sources of catalytic reagents for reactions required to liberate the biomolecules of interest.

Certain embodiments of the inventive process described herein use animal viscera (defined herein as the internal organs in the main cavity of an animal body) as a source of digestive enzymes for assisting in hydrolysis of the animal by-products. In one embodiment, the hydrolysis benefits from addition of acid which may be an organic acid or a mineral acid to chemically lower the pH of the hydrolysis mixture as required. In another embodiment, the hydrolysis benefits from supplementation with fermentation by acid-producing bacteria in a growth medium containing an appropriate energy source for the bacteria such as a carbohydrate. The bacteria produce organic acids which lower the pH of the mixture and promote hydrolysis. In each embodiment, digestive enzymes are activated to degrade the matrices of the animal by-products and release the desired biomolecules, such as glycosaminoglycans, collagen, peptides, ossein and oils.

Embodiments of the present invention are capable of producing a plurality of different classes of bioactive biomolecules from waste by-products of fish, poultry without a need for a defatting step or alkaline and thermal pre-treatments as required by the current processing techniques. The energy consumption is minimal, as 37° C. is the optimum temperature for the enzymatic digestion step described herein.

Various aspects of the invention will now be described with reference to the figures. A number of possible alternative features are introduced during the course of this description. It is to be understood that, according to the knowledge and judgment of persons skilled in the art, such alternative features may be substituted in various combinations to arrive at different embodiments of the present invention.

### Embodiment 1: Acid-Assisted Hydrolysis

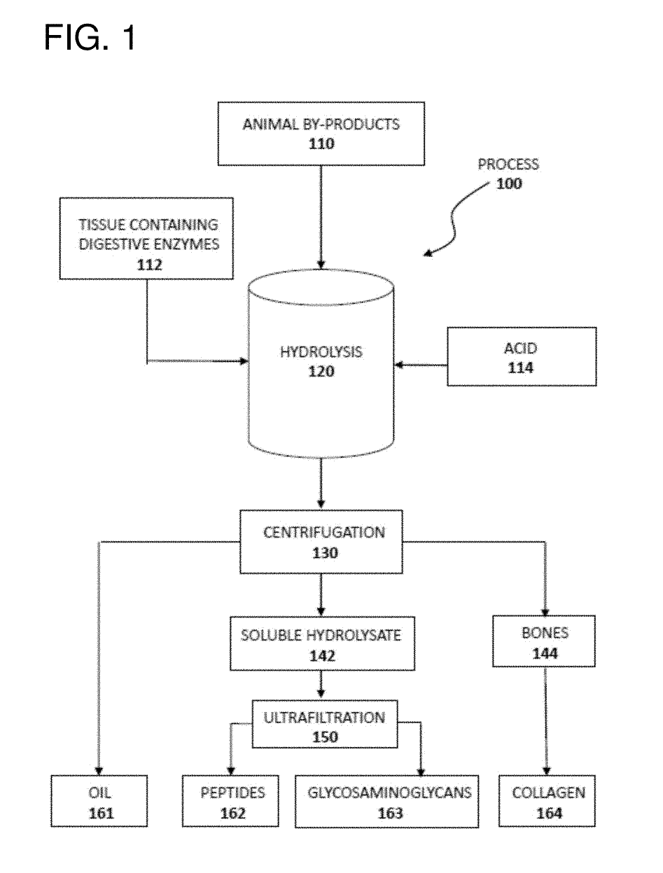

FIG. 1 illustrates a first embodiment 100 of general process of isolation of four different classes of biomolecules from animal by-products 110 typically considered as waste materials. Such animal by-products typically represent left over parts after the primary meat products are removed. Examples of such animal by-products include skins, cartilage, connective tissue, appendages and bones from animals such as fish, poultry and mammals such as pigs and cattle. The animal by-products 110 are mixed with tissues containing digestive enzymes 112 in the form of differentiated or undifferentiated animal organs, typically visceral organs, obtained from fish, poultry and mammals such as pigs and cattle. An acid 114 is added to decrease the pH of the mixture and promote degradation of the tissues containing digestive enzymes 112 to promote hydrolysis 120 to release the desired bio-active biomolecules from their tissue matrices.

When the hydrolysis step is deemed complete, the hydrolysis mixture is subjected to centrifugation 130 to generate different fractions including an oil 161 which may be considered a biomolecule product mixture, a solid fraction comprising bones 144 which are processed further to generate collagen 164. Gelatin or ossein may also be isolated from the bones 144.

There is also a liquid fraction of a soluble hydrolysate 142 which is subjected to ultrafiltration 150 to produce peptides 162 and glycosaminoglycans 163.

### Embodiment 2: Fermentation-Assisted Hydrolysis with Acid-Producing Bacteria

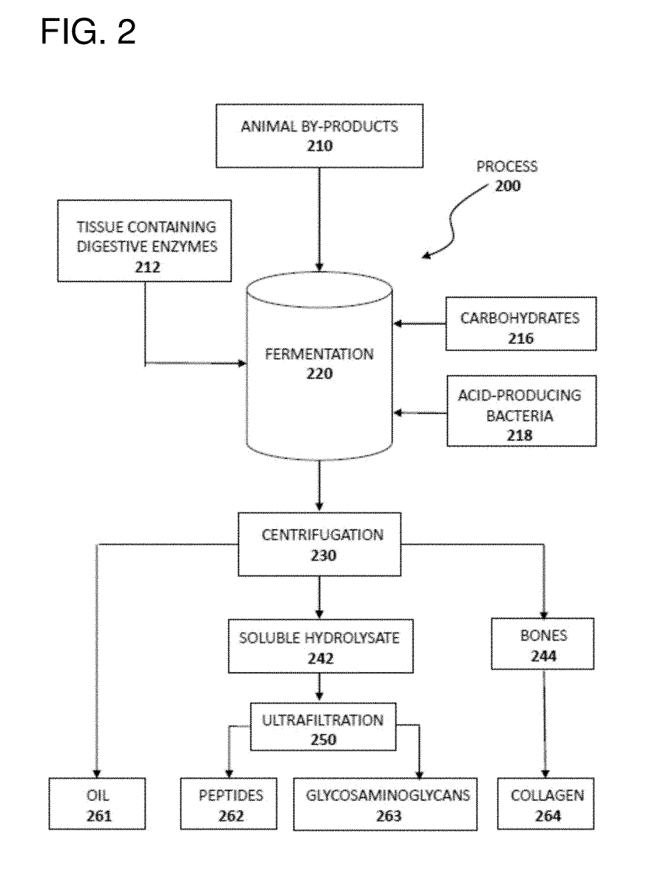

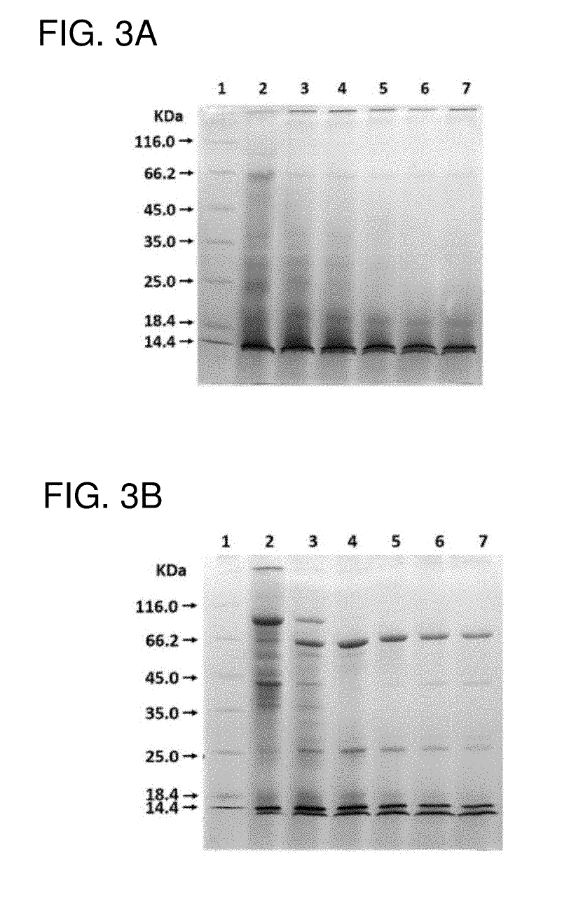

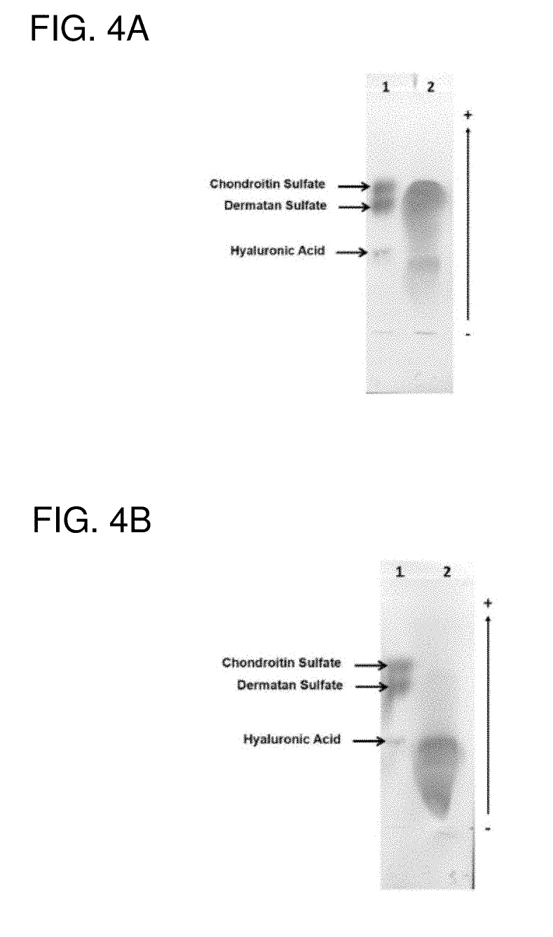

FIG. 2 illustrates a second embodiment 200 of general process of isolation of four different classes of biomolecules from animal by-products 210 typically considered as waste materials, as described in Example 1. As described in Example 1, the animal by-products 210 are mixed with tissues containing digestive enzymes 212, typically in the form of differentiated or undifferentiated animal organs, typically visceral organs, obtained from fish, poultry and mammals such as pigs and cattle. Instead of addition of an organic or mineral acid, this embodiment includes the steps of addition of acid-producing bacteria 218 and carbohydrates 216 as an energy source to promote growth and acid production by the acid-producing bacteria during fermentation 220. This step is ideally conducted in a fermenter which may be configured specifically to accept and process the animal by-products and tissues with sufficient mixing as required.

When the fermentation step 200 is deemed complete, the fermented mixture is subjected to centrifugation 230 as described in Example 1, to generate different fractions including an oil 261 which may be considered a biomolecule product mixture, a solid fraction comprising bones 244 which are processed further to generate collagen 264. Gelatin may also be isolated from the bones 244.

As described above for Example 1, there is also a liquid fraction of a soluble hydrolysate 242 which is subjected to ultrafiltration 250 to produce peptides 262 and glycosaminoglycans 263.

### Examples

### Example 1: Acid-Assisted Hydrolysis

In this example, 2 parts (by weight) of glycosaminoglycan/protein rich material (e.g. poultry heads, fish heads, and bone biomass after mechanical deboning of meat) is mixed with 1 part (by weight) of tissues containing digestive enzymes (e.g. fish viscera) then ground and homogenized. After which, 2% (by weight) of organic or mineral acid (e.g. formic acid) is added to the mixture and incubated at 37° C. for 3 days under continuous mixing.

### Example 2: Hydrolysis with Microbial-Assisted Fermentation

In this example, 2 parts (by weight) of glycosaminoglycan/protein rich material (e.g. poultry heads, fish heads, bone biomass after mechanical deboning of meat) is mixed with 1 part (by weight) of tissues containing digestive enzymes (e.g. fish viscera) then ground and homogenized. After which, 10% (by weight) of fermentable sugar (e.g. powdered lactose or dairy whey permeate) and 1% (by weight) of acid producing bacteria are added to the mixture. The mixture is incubated at 37° C. for 3 days under continuous mixing. Any strain of a lactic acid bacterium may be used. In this example, the inoculum contains two such strains, Lactobacillus plantarum and Pediococcus acidilactici.

### Example 3: Recovery of Biomolecules

After hydrolysis, the mixture is heated (e.g. 80° C. for 10 min) to inactivate the enzymes. The solution is then passed through a sieve to separate the partially decalcified bone (ossein) from the rest of the mixture. The ossein is washed and can further be used to produce gelatin after further demineralization and extraction in warm water. The liquid part is centrifuged (e.g. 10,000×g for 15 minutes) which will result in 3 fractions: an oil/fat fraction in the top layer, a soluble collagen peptides/glycosaminoglycan fraction in the middle layer and an unhydrolyzed residue fraction in the bottom layer.

The fat layer can be purified by addition of sodium sulfate which will absorb the residual and trap the impurities. The middle layer is then subjected to ultrafiltration (using a 10 kDa cut-off membrane) to separate the collagen peptides (which have a molecular weight <10 kDa) from the glycosaminoglycans (which have a molecular weight >10 kDa). The soluble collagen peptides can further be dried using a drying method. The soluble glycosaminoglycans can be precipitated using a polar solvent (such as 70% ethanol) and then dried. Non-sulfated glycosaminoglycans (e.g. hyaluronic acid) can be separated from sulfated glycosaminoglycans (e.g. chondroitin sulfate, dermatan sulfate, keratan sulfate) using anion exchange chromatography. The isolation of individual sulfated glycosaminoglycans can be achieved through selective precipitation using a polar solvent (such as ethanol) at increasing concentrations.

### EQUIVALENTS AND SCOPE

Other than described herein, or unless otherwise expressly specified, all of the numerical ranges, amounts, values and percentages, such as those for amounts of materials, elemental contents, times and temperatures, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Any patent, publication, internet site, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Unless otherwise defined, all technical and scientific terms used herein have the same meaning as commonly understood by one of ordinary skill in the art to which this invention belongs.

While this invention has been particularly shown and described with references to embodiments thereof, it will be understood by those skilled in the art that various changes in form and details may be made therein without departing from the scope of the invention encompassed by the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context.

It is also noted that the term “comprising” is intended to be open and permits but does not require the inclusion of additional elements or steps. When the term “comprising” is used herein, the term “consisting of” is thus also encompassed and disclosed. Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where the term “about” is used, it is understood to reflect+/−10% of the recited value. In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein.

1. A process for producing a plurality of biomolecule products from by-products of animal food processing, the process comprising:
mixing the by-products with one or more digestive enzymes in the presence of an acid to promote hydrolysis of the by-product to release the biomolecules, thereby providing a hydrolysis mixture;
subjecting the hydrolysis mixture to a density-based fractional separation, thereby providing an oil fraction, a liquid fraction and a solid fraction; and
separating the liquid fraction from the oil and solid fractions and filtering the liquid fraction with a molecular mass cutoff filter, thereby providing a peptide product and a glycosaminoglycan product.

2. The process of claim 1, further comprising processing bone tissue contained in the solid fraction to generate one or more of or a combination of: a collagen product, a gelatin product and an ossein product.

3. The process of claim 1, further comprising processing the oil to provide a refined oil product.

4. The process of claim 1, wherein the step of mixing the by-products further includes homogenization of the by-products.

5. The process of claim 1, wherein the digestive enzymes are provided in the form of substantially intact biological tissues.

6. The process of claim 5, wherein the biological tissues are of substantially intact organs.

7. The process of claim 6, wherein the substantially intact organs are digestive tract organs, pancreas, liver and salivary glands.

8. The process of claim 5, wherein the digestive enzymes are endogenous proteases, peptidases, amylases, lipases and nucleases.

9. The process of claim 5, wherein the biological tissues comprise undifferentiated viscera.

10. The process of claim 9, wherein the undifferentiated viscera comprise any one of or any combination of: digestive tract organs, pancreas, liver and salivary glands.

11. The process of claim 1, wherein the animal by-products comprise any one of or any combination of: bones, skin, organs, cartilage, connective tissues and appendages from fish, poultry or mammals.

12. The process of claim 1, wherein the acid is an organic acid or a mineral acid which is directly added to obtain the hydrolysis mixture.

13. The process of claim 1, wherein the acid is produced during fermentation by one or more species or strains of acid-producing bacteria.

14. The process of claim 13, wherein the bacteria are lactic acid-producing bacteria.

15. The process of claim 13, which is supported by addition of a carbohydrate to promote acid production by the bacteria.

16. The process of claim 1, wherein the density-based fractional separation is centrifugation.

17. The process of claim 1, wherein the molecular mass cutoff filter has a molecular mass cutoff at between about 5 kDa to about 15 kDa.

18. The process of claim 1, wherein the glycosaminoglycan product comprises any one of or a combination of: hyaluronic acid, chondroitin sulfate, dermatan sulfate and keratan sulfate.

19. A process for producing a plurality of biomolecule products from by-products of animal food processing, the process comprising:
mixing the by-products with undifferentiated viscera in the presence of an acid to promote hydrolysis of the by-product to release the biomolecules, thereby providing a hydrolysis mixture;
subjecting the hydrolysis mixture to a density-based fractional separation, thereby providing an oil fraction, a liquid fraction and a solid fraction; and
separating the liquid fraction from the oil and solid fractions and filtering the liquid with a molecular mass cutoff filter, thereby providing a peptide product and a glycosaminoglycan product.

20. The process of claim 19, wherein the undifferentiated viscera are fish viscera.
